(19) United States
(12) Patent Application Publication
(10) Pub. No.: US 2022/0415453 A1
(43) Pub. Date: Dec. 29, 2022

(54) DETERMINING A DISTRIBUTION OF ATOM COORDINATES OF A MACROMOLECULE FROM IMAGES USING AUTO-ENCODERS

(71) Applicant: DeepMind Technologies Limited, London (GB)

(72) Inventors: Olaf Ronneberger, London (GB); Marta Garnelo Abellanas, London (GB); Dan Rosenbaum, London (GB); Seyed Mohammadali Eslami, London (GB); Jonas Anders Adler, London (GB)

(21) Appl. No.: 17/849,269

(22) Filed: Jun. 24, 2022

Related U.S. Application Data
(60) Provisional application No. 63/215,357, filed on Jun. 25, 2021.

(51) Int. Cl. G16C 20/70; G16C 20/80

(52) U.S. Cl. CPC G16C 20/70; G16C 20/80; G06N 3/088

(57) ABSTRACT

Methods, systems and apparatus, including computer programs encoded on computer storage media. One of the methods includes obtaining a plurality of images of a macromolecule having a plurality of atoms, training a decoder neural network on the plurality of images, and after the training, generating a plurality of conformations for at least a portion of the macromolecule that each include respective three-dimensional coordinates of each of the plurality of atoms, wherein generating each conformation includes sampling a conformation latent representation from a prior distribution over conformation latent representations, processing a respective input including the sampled conformation latent representation using the decoder neural network to generate a conformation output that specifies three-dimensional coordinates of each of the plurality of atoms for the conformation, and generating the conformation from the conformation output.

### CROSS-REFERENCE TO RELATED APPLICATION

This application claims priority to U.S. Provisional Application No. 63/215,357, filed on Jun. 25, 2021. The disclosure of the prior application is considered part of and is incorporated by reference in the disclosure of this application.

### BACKGROUND

This specification relates to processing images of a macromolecule, e.g., a protein, using neural networks.

A protein is specified by one or more sequences (“chains”) of amino acids. An amino acid is an organic compound which includes an amino functional group and a carboxyl functional group, as well as a side chain (i.e., group of atoms) that is specific to the amino acid. Protein folding refers to a physical process by which one or more sequences of amino acids fold into a three-dimensional (3-D) configuration. The structure of a protein defines the 3-D configuration of the atoms in the amino acid sequences of the protein after the protein undergoes protein folding. When in a sequence linked by peptide bonds, the amino acids may be referred to as amino acid residues or simply residues.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current value inputs of a respective set of parameters.

### SUMMARY

This specification describes a system implemented as computer programs on one or more computers in one or more locations that determines a distribution of atom coordinates of a macromolecule, e.g., a protein, from images of the macromolecule, e.g., cryogenic electron microscopy (cryo-EM) images of the macromolecule. In other words, the system determines a plurality of conformations for the macromolecule. Each conformation specifies the three-dimensional coordinates of a plurality of atoms in the macromolecule in some fixed coordinate system. As a particular example, each conformation can represent the residues in a protein as a rigid body and specify the coordinates of each atom in each of a plurality of residues.

As used throughout this specification, the term “protein” can be understood to refer to any biological molecule that is specified by one or more sequences (or “chains”) of amino acids. For example, the term protein can refer to a protein domain, e.g., a portion of an amino acid chain of a protein that can undergo protein folding nearly independently of the rest of the protein. As another example, the term protein can refer to a protein complex, i.e., that includes multiple amino acid chains that jointly fold into a protein structure.

According to a first aspect, there is provided a method performed by one or more computers, the method comprising: obtaining a plurality of images of a macromolecule having a plurality of atoms; training a decoder neural network on the plurality of images, wherein the decoder neural network is configured to receive an input comprising a conformation latent representation of a conformation of the macromolecule and to process the input to generate a conformation output that specifies three-dimensional coordinates of each of the plurality of atoms; and after the training, generating a plurality of conformations for at least a portion of the macromolecule that each include respective three-dimensional coordinates of each of the plurality of atoms, comprising, for each conformation: sampling a conformation latent representation from a prior distribution over conformation latent representations; processing a respective input comprising the sampled conformation latent representation using the decoder neural network to generate a conformation output that specifies three-dimensional coordinates of each of the plurality of atoms for the conformation; and generating the conformation from the conformation output.

In some implementations, the conformation output specifies, for each of the plurality of atoms, a respective delta for base three-dimensional coordinates for the atom in a base conformation for the macromolecule.

In some implementations, generating the conformation from the conformation output comprises: for each of the plurality of atoms, applying the respective delta specified by the conformation output for the atom to the base three-dimensional coordinates for the atom to generate the respective three-dimensional coordinates for the atom.

In some implementations, the method further comprises: determining the base conformation for the macromolecule through a single state reconstruction.

In some implementations, the delta specifies, for each of a plurality of residues that each include one or more of the plurality of atoms, a respective relative translation and relative rotation for the residue relative to a position of the residue in the base conformation.

In some implementations, training the decoder neural network on the plurality of images comprises: training the decoder neural network jointly with an encoder neural network that is configured to receive an image of the macromolecule and to process the image to generate an encoder output that comprises parameters of a posterior distribution over the conformation latent representations.

In some implementations, training the decoder neural network jointly with the encoder neural network comprises: obtaining a batch of one or more images from the plurality of images; for each image in the batch: processing the image using the encoder neural network to generate an encoder output; sampling a set of conformation latent representations from the posterior distribution in accordance with the parameters of the posterior distribution in the encoder output; processing each of the conformation latent representations in the set using the decoder neural network to generate a respective decoder output for each of the conformation latent representations; generating a respective reconstruction of the image from each of the decoder outputs using a differentiable renderer; and training the encoder neural network and the decoder neural network on a loss function that includes one or more loss terms that measure, for each image in the batch, an error between the image and the respective reconstructions of the image generated from the decoder output for the image.

In some implementations, the set of conformation latent representations includes a plurality of conformation latent representations.

In some implementations, the loss function includes one or more auxiliary loss terms that measure, for each decoder output, a deviation of a structure of the macromolecule as specified by the three-dimensional coordinates of each of the plurality of atoms from an expected structure of the macromolecule.

In some implementations, the auxiliary loss terms include a first auxiliary loss term that measures a deviation between (i) bond lengths along a backbone of the macromolecule in the structure specified by the three-dimensional coordinates of each of the plurality of atoms and (ii) expected bond lengths along the backbone of the macromolecule.

In some implementations, the auxiliary loss terms include a second auxiliary loss term that measures a deviation between (i) a center of mass of the structure specified by the three-dimensional coordinates of each of the plurality of atoms and (ii) an expected center of mass of the structure.

In some implementations, the loss function includes one or more terms that measure, for each encoder output, a divergence between the posterior distribution and the prior distribution in accordance with the parameters specified in the encoder output.

In some implementations, the encoder neural network is configured to process the image to generate an encoded representation of the image and to process the encoded representation of the image to generate the parameters of the posterior distribution over the conformation latent representations; the encoder neural network is configured to process at least the encoded representation to generate parameters of a posterior distribution over pose latent representations; and generating a respective reconstruction of the image from each of the decoder outputs using a differentiable renderer comprises: sampling a pose latent representation from the posterior distribution over pose latent representations in accordance with the parameters of the posterior distribution; and for each decoder output, generating the respective reconstruction of the image using the sampled pose latent representation and the differentiable renderer.

In some implementations, generating the respective reconstruction of the image using the sampled pose latent representation and the differentiable renderer comprises: generating, from the decoder output, three-dimensional coordinates of each of the plurality of atoms; modifying a pose of the plurality of atoms using the sampled pose latent to generate modified three-dimensional coordinates of each of the plurality of atoms; and applying the differentiable renderer to the modified three-dimensional coordinates of each of the plurality of atoms to generate the respective reconstruction.

In some implementations, the decoder neural network is configured to process the input to generate a decoded representation of the conformation latent representation and to process the decoded representation to generate the decoder output, and wherein the encoder neural network is configured to process the encoded representation of the image and respective decoded representation of each conformation latent representation in the set to generate the parameters of the posterior distribution over pose latent representations.

In some implementations, the plurality of images are Cryo-electron microscopy (cryo-EM) images of the macromolecule.

In some implementations, the plurality of images are picked particle images of the macromolecule.

In some implementations, the macromolecule is a protein.

According to another aspect, there is provided a method of imaging different conformations of a macromolecule by processing a cryogenic electron microscopy image comprising a plurality of images of the macromolecule, to determine a plurality of conformations of a structure of the macromolecule, the method comprising: obtaining at least one cryogenic electron microscope image comprising the plurality of images of the macromolecule; and generating the plurality of conformations for at least a portion of the macromolecule; wherein the plurality of conformations represents different conformations of the structure of at least the portion of macromolecule, and wherein the plurality of images of the macromolecule comprises images of the macromolecule frozen in the plurality of different conformations.

According to another aspect there is provided a system comprising one or more computers and one or more storage devices storing instructions that when executed by the one or more computers cause the one more computers to perform the operations of the methods described herein.

According to another aspect, there are provided one or more computer storage media storing instructions that when executed by one or more computers cause the one more computers to perform the operations of the methods described herein.

According to another aspect, there is provided a cryogenic electron microscopy system comprising: an electron microscope to capture an image of a cryogenic electron microscopy specimen, wherein the specimen comprises a plurality of examples of a macromolecule frozen in different conformations, and wherein the captured image comprises images of the macromolecule frozen in the different conformations; and a computer system configured to process the captured image to determine the plurality of conformations representing the different conformations of the structure of the frozen macromolecule.

According to another aspect there is provided a non-transitory computer storage medium storing data that defines a plurality of conformations for at least a portion of a macromolecule, wherein the data was generated by operations of the methods described herein.

The protein structure prediction system (conformation prediction system) described herein can be used to obtain a ligand such as a drug or a ligand of an industrial enzyme. For example, a method of obtaining a ligand may include obtaining a set of images (e.g., cryo-EM images) of a target protein, and processing images using the protein structure prediction system to generate one or more conformations of the target protein, i.e., that each define a respective predicted structure of the target protein. The method may then include evaluating an interaction of one or more candidate ligands with each predicted structure of the target protein. The method may further include selecting one or more of the candidate ligands as the ligand dependent on a result of the evaluating of the interaction.

In some implementations, evaluating the interaction may include evaluating binding of the candidate ligand with each predicted structure of the target protein. For example, evaluating the interaction may include identifying a ligand that binds with sufficient affinity for a biological effect. In some other implementations, evaluating the interaction may include evaluating an association of the candidate ligand with each predicted structure of the target protein which has an effect on a function of the target protein, e.g., an enzyme. The evaluating may include evaluating an affinity between the candidate ligand and each predicted structure of the target protein, or evaluating a selectivity of the interaction.

The candidate ligand(s) may be derived from a database of candidate ligands, and/or may be derived by modifying ligands in a database of candidate ligands, e.g., by modifying a structure or amino acid sequence of a candidate ligand, and/or may be derived by stepwise or iterative assembly/optimization of a candidate ligand.

The evaluation of the interaction of a candidate ligand with a predicted structure of the target protein may be performed using a computer-aided approach in which graphical models of the candidate ligand and target protein structure are displayed for user-manipulation, and/or the evaluation may be performed partially or completely automatically, for example using standard molecular (protein-ligand) docking software. In some implementations the evaluation may include determining an interaction score for the candidate ligand, where the interaction score includes a measure of an interaction between the candidate ligand and the target protein. The interaction score may be dependent upon a strength and/or specificity of the interaction, e.g., a score dependent on binding free energy. A candidate ligand may be selected dependent upon its score.

In some implementations the target protein includes a receptor or enzyme and the ligand is an agonist or antagonist of the receptor or enzyme. In some implementations the method may be used to identify the structure of a cell surface marker. This may then be used to identify a ligand, e.g., an antibody or a label such as a fluorescent label, which binds to the cell surface marker. This may be used to identify and/or treat cancerous cells.

In some implementations the candidate ligand(s) may include small molecule ligands, e.g., organic compounds with a molecular weight of <900 daltons. In some other implementations the candidate ligand(s) may include polypeptide ligands, i.e., defined by an amino acid sequence.

In some cases, the protein structure prediction system can be used to determine one or more predicted structures of a candidate polypeptide ligand, e.g., a drug or a ligand of an industrial enzyme. The interaction of this with a target protein structure may then be evaluated; the target protein structure may have been determined using a structure prediction neural network or using conventional physical investigation techniques such as x-ray crystallography and/or magnetic resonance techniques.

Thus in another aspect there is provided a method of using a protein structure prediction system to obtain a polypeptide ligand (e.g., the molecule or its sequence). The method may include obtaining a respective set of images (e.g., cryo-EM images) of each of one or more candidate polypeptide ligands. The method may further include using the protein structure prediction system to determine conformations defining (tertiary) structures of the candidate polypeptide ligands. The method may further include obtaining a target protein structure of a target protein, in silico and/or by physical investigation, and evaluating an interaction between the structure of each of the one or more candidate polypeptide ligands and the target protein structure. The method may further include selecting one or more of the candidate polypeptide ligands as the polypeptide ligand dependent on a result of the evaluation.

As before evaluating the interaction may include evaluating binding of the candidate polypeptide ligand with the structure of the target protein, e.g., identifying a ligand that binds with sufficient affinity for a biological effect, and/or evaluating an association of the candidate polypeptide ligand with the structure of the target protein which has an effect on a function of the target protein, e.g., an enzyme, and/or evaluating an affinity between the candidate polypeptide ligand and the structure of the target protein, or evaluating a selectivity of the interaction. In some implementations the polypeptide ligand may be an aptamer.

Implementations of the method may further include synthesizing, i.e., making, the small molecule or polypeptide ligand. The ligand may be synthesized by any conventional chemical techniques and/or may already be available, e.g., may be from a compound library or may have been synthesized using combinatorial chemistry.

The method may further include testing the ligand for biological activity in vitro and/or in vivo. For example the ligand may be tested for ADME (absorption, distribution, metabolism, excretion) and/or toxicological properties, to screen out unsuitable ligands. The testing may include, e.g., bringing the candidate small molecule or polypeptide ligand into contact with the target protein and measuring a change in expression or activity of the protein.

In some implementations a candidate (polypeptide) ligand may include: an isolated antibody, a fragment of an isolated antibody, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. A candidate (polypeptide) ligand may include an antibody with a mutated or chemically modified amino acid Fc region, e.g., which prevents or decreases ADCC (antibody-dependent cellular cytotoxicity) activity and/or increases half-life when compared with a wild type Fc region.

Misfolded proteins are associated with a number of diseases. Thus in a further aspect there is provided a method of using the protein structure prediction system to identify the presence of a protein mis-folding disease. The method may include obtaining a set of images (e.g., cryo-EM images) of a protein and using the protein structure prediction system to determine one or more conformations (structures) of the protein. The method may further include obtaining a structure of a version of the protein obtained from a human or animal body, e.g., by conventional (physical) methods. The method may then include comparing the structure of the protein with the structure of the version obtained from the body and identifying the presence of a protein mis-folding disease dependent upon a result of the comparison. That is, mis-folding of the version of the protein from the body may be determined by comparison with the in silico determined structure.

In some other aspects a computer-implemented method as described above or herein may be used to identify active/binding/blocking sites on a target protein from a set of images (e.g., cryo-EM images) of the target protein.

The system can determine the distribution of atom coordinates of a macromolecule by obtaining a plurality of images of the macromolecule having a plurality of atoms.

The system can then train a decoder neural network on the plurality of images, e.g., as part of a variational auto-encoder training framework or otherwise. The decoder neural network is configured, in particular trained, to receive an input comprising a conformation latent representation of a conformation of the macromolecule and to process the input to generate a conformation output that specifies three-dimensional coordinates of each of the plurality of atoms.

After the training, the system generates a plurality of conformations for at least a portion of the macromolecule that each include respective three-dimensional coordinates of each of the plurality of atoms.

To generate a conformation after training, the system samples a conformation latent representation from a prior distribution over conformation latent representations, e.g., a normal distribution over conformation latent representations.

The system then processes a respective input comprising the sampled conformation latent representation using the decoder neural network to generate a conformation output that specifies three-dimensional coordinates of each of the plurality of atoms for the conformation.

The system then generates the conformation from the conformation output.

Historically x-ray crystallography has been used to determine the structures of macromolecules. However this locks the macromolecules into a crystalline lattice. By contrast cryo-EM can capture the different conformations that a molecule can have in its natural environment. For example, many macromolecules can adopt different conformations, e.g., to perform particular biological functions. More generally, they may deform in an aqueous environment. As another example a protein may change shape when it binds to a ligand. It is therefore useful to be able to determine the different configurations of, or a distribution of the configurations of, a macromolecule, but this is a difficult computational problem.

Thus there is also described a method of imaging different conformations of a molecule, in particular a macromolecule, by processing at least one cryogenic electron microscopy image comprising a plurality of images of the macromolecule. The method can determine a plurality of conformations, e.g., a distribution of conformations, of a structure of the macromolecule. These represent the different conformations of, e.g., the distribution of conformations of, the macromolecule in the cryogenic electron microscopy image(s).

The method involves obtaining at least one cryogenic electron microscope image comprising a plurality of images of the macromolecule, from an electron microscope. In some cryo-EM implementations each image of the macromolecule is a projection image of the macromolecule. The plurality of images of the macromolecule comprises images of the macromolecule frozen (at different lateral locations in the image) in the plurality of different conformations.

The above described method is used to obtain the plurality of conformations for at least a portion of the macromolecule, where the plurality of conformations represents different conformations of at least the portion of the structure of the frozen macromolecule.

There is also described a cryogenic electron microscopy system. The system comprises an electron microscope, e.g., a transmission electron microscope (TEM), to capture an image of a cryogenic electron microscopy (cryo-EM) specimen. In general a cryo-EM specimen may comprise a film of a frozen solution of macromolecules on a substrate. Thus the specimen generally comprises a plurality of examples of a macromolecule frozen in different conformations, and the captured image comprises images of the macromolecule frozen in the different conformations.

The cryogenic electron microscopy system includes a computer system, configured to process the captured image(s) as described above to determine the plurality of conformations representing the different conformations of the structure of the frozen macromolecule.

Data representing a result of imaging the different conformations of the macromolecule may be provided in any convenient manner, e.g., as one or more images, or as a video, or as a data file that may be stored or transmitted for subsequent use. The result of the imaging need not explicitly represent the coordinates of each atom; for example a protein structure may be represented as a ribbon diagram.

Particular embodiments of the subject matter described in this specification can be implemented so as to realize one or more of the following advantages.

Imaging of macromolecules (e.g., cryo-EM imaging of proteins) has enabled significant advances in macromolecule structure determination. However, many imaging techniques provide either a single state of the studied macromolecule, or a relatively small number of its conformations. Thus many imaging techniques fail to capture the full range of possible conformations of macromolecules with flexible regions. The system described in this specification provides a deep-learning based approach for generating a continuous distribution of atomic macromolecule structures directly from a set of macromolecule images.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF DRAWINGS

FIG. 1 depicts an example conformation prediction system that can determine multiple conformations of a macromolecule.

FIG. 2A and FIG. 2B are schematic diagrams of an example training engine that can jointly train a decoder and encoder neural network as part of variational autoencoder framework.

FIG. 3 shows an example generating engine that can cause a decoder neural network to generate conformations of a macromolecule.

FIG. 4 is a flow diagram of an example process for generating multiple conformations of a macromolecule from a decoder neural network.

FIG. 5 is a flow diagram of an example process for jointly training a decoder neural network and an encoder neural network.

FIG. 6A shows parameters of a cryo-EM simulator used in experimentation.

FIG. 6B shows examples of picked particle images used in experimentation.

FIG. 6C shows examples of determined conformations of AurA.

FIG. 7A is a sample of experimental data illustrating a predicted distribution vs. a ground truth distribution.

FIG. 7B is a sample of experimental data comparing predicted vs. ground truth proportionalities of macromolecule states.

Like reference numbers and designations in the various drawings indicate like elements.

### DETAIL DESCRIPTION

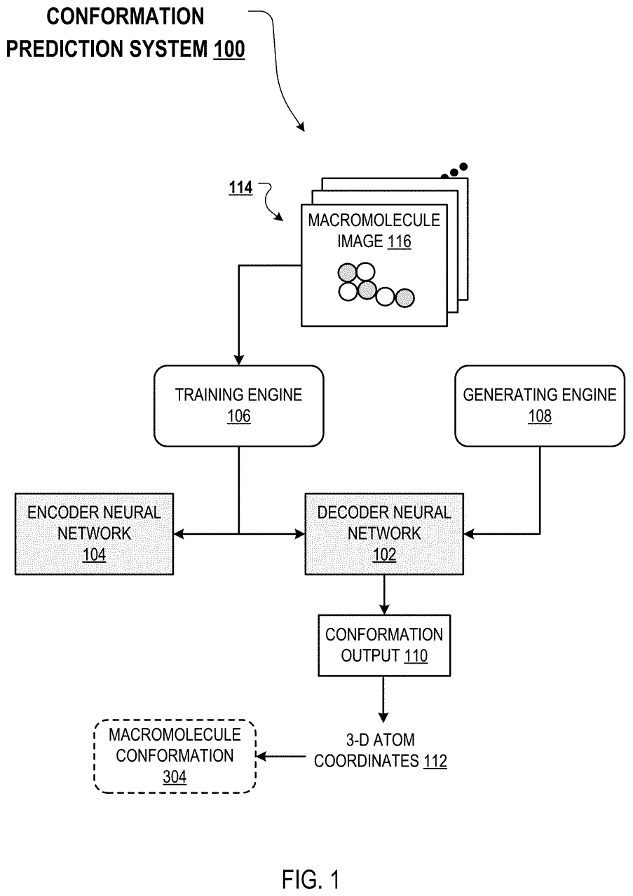

FIG. 1 depicts an example conformation prediction system 100 that can determine multiple conformations of a macromolecule using a variational autoencoder and a set of images. The conformation prediction system 100 is an example of a system implemented as computer programs on one or more computers in one or more locations in which the systems, components, and techniques described below are implemented.

The system 100 obtains a set of images 114, e.g., electron microscope images, of a macromolecule. Macromolecules are large molecules (e.g., proteins, nucleic acids, or polymers) that can be important to biochemical processes. For example, a macromolecule may be a molecule with a molecular weight of >1000 Da. In the context of cryo-EM such a macromolecule may also be referred to as a particle.

The macromolecule is composed of constituent atoms (in some cases, thousands of atoms) arranged in a spatial conformation based on covalent bonding. Since groups of atoms (e.g., residues) can rotate freely about single bonds, and more generally can flex, twist, and deform, the macromolecule can be in any one of multiple possible conformations, in general in a continuous distribution of conformations. For example, a protein can have flexible regions that permit protein folding, creating complex inter-residue movement and deformations of the protein structure.

A single macromolecule image 116 depicts the macromolecule in one of these possible spatial conformations, and in a particular macromolecule configuration. A particular macromolecule configuration in the, e.g., continuous distribution of configurations can be identified with a conformation and a pose. The conformation specifies, in some fixed coordinate system, three-dimensional (3-D) coordinates of each of the constituent atoms in the macromolecule. Conformations defined in this fixed coordinate system are unique macromolecule structures that cannot be translated or rotated into one another.

In some cases, multiple samples of the macromolecule are prepared to acquire the set of images 116. Each image 116 in the set 114 usually shows the macromolecule in a slightly different configuration. In cryo-EM a single cryogenic electron microscopy image, sometimes called a micrograph, typically comprises a plurality of images of the macromolecule frozen in different conformations. The system 100 can utilize this inherent heterogeneity to reconstruct a distribution of these configurations, e.g., a continuous distribution, using only a limited number of configurations portrayed in the set of images 114.

In some cases, the 3-D atom coordinates are defined relative to 3-D coordinates of their associated residues, i.e., the particular group of atoms the atom belongs to. Residues can be described as rigid bodies where relative positions of atoms in the residue are fixed with respect to one another. Hence, conformations can be specified by their residue coordinates (and orientations) and the atom coordinates are inferred from the residue coordinates.

Alternatively or in addition, conformations can be defined relative to a base, or reference, conformation which, e.g., specifies some known conformation of the macromolecule. The base conformation can be obtained by single state reconstruction of the macromolecule or computationally modelling the macromolecule, foregoing an experimentally determined structure or template. In some cases, the base conformation can be obtained from a data bank such as the Protein Data Bank. In some implementations of the method conformations are defined by differences or “deltas” that describe relative translation and rotation of each residue of the macromolecule with respect to the base, or reference, conformation. For example, a macromolecule conformation may specify, for each of a plurality of atoms in the macromolecule, a respective delta for base three-dimensional coordinates for the atom in a base conformation for the macromolecule, i.e., where the delta specifies three-dimensional coordinates for the atom with respect to a three-dimensional position for the atom in the base or reference conformation of the macromolecule. This can be a practical approach when generating conformations computationally as the base conformation provides a useful structure to manipulate into different conformations.

On the other hand, the pose specifies an orientation of a conformation in a global coordinate system, where the fixed coordinate system in which the macromolecules conformations are specified may be translated and/or rotated with respect to the global coordinate system. Consequently, the pose corresponds to a global translation and/or rotation of all the collective atoms/residues constituting the macromolecule.

Since poses of conformations are usually random when being imaged, a single conformation may be imaged from multiple perspectives. This can be a significant problem when imaging large, complex macromolecules as the structure of a conformation may appear vastly different from different perspectives. Hence, a single conformation viewed from multiple perspectives may be misinterpreted as multiple conformations. As will be described later, the system 100 disentangles conformations and poses to predict unique macromolecule structures.

Accordingly, the macromolecule image 116 represents a two-dimensional (2-D) projection of the 3-D macromolecule in a specific conformation and pose. The system 100 reconstructs the 3-D conformations of the macromolecule using these various 2-D projections. Provided with a sufficient number of sampled conformations in different poses, the system 100 can determine multiple conformations of a macromolecule, e.g., a continuous distribution of conformations.

In some implementations, the set of images 114 are obtained from cryogenic electron microscopy (cryo-EM) data. Tens of thousands to millions of highly noisy macromolecule images 116 can be generated by cryo-EM, where an image comprising many individual macromolecule images is usually referred to as a “micrograph”. The set of images 114 may be obtained from one or more micrographs. The system 100 can compensate for noise in the set of images 114 by computationally combining information from all such images and utilizing correlations between various images. The set of images 114 may comprise picked particle images of the macromolecule. A picked particle image can be an image of a macromolecule extracted from a micrograph, e.g., manually or automatically, e.g., by any of a range of standard techniques. In general the aim is to pick out a single particle (macromolecule); the picked particle image may be preprocessed, e.g., to isolate the picked particle from contaminants or other particles. In general, the set of images 114 can be obtained from any suitable type of molecular imaging data.

In some implementations, e.g., to verify correct operation of the system, the set of images 114 are obtained from simulated data. Realistic simulated data can be employed by the system 100 for effective training and experimentation. For example, an externally validated Molecular Dynamics (MD) simulation and a high quality image formation simulator including, e.g., a contrast transfer function, realistic noise models and solvation, can generate the set of images 114 for various conformations and poses of the macromolecule. The system 100 can then be deployed on real world data captured in real images of a macromolecule. In some cases, real world data is supplemented with simulated data to provide the system 100 with more information on the possible configurations of the macromolecule, leading to improved performance of the system 100.

The system 100 trains a decoder neural network 102 on the set of images 114 using a training engine 106. In some implementations, the training engine 106 trains the decoder neural network 102 jointly with an encoder neural network 104, e.g., as part of a variational autoencoder (VAE) training framework; in these configurations the encoder neural network 104 is not needed after training. The training engine 106 can be configured to perform various operations that are necessary during training of the neural networks, as will be described in detail below. The system 100 can be a fully “end-to-end” machine learning approach when implementing the VAE training framework. In an end-to-end implementation, the system 100 uses the neural networks to perform all tasks involved in determining the conformations of the macromolecule from the set of images 114. No processing, such as feature extraction, is performed by a separate system. Nonetheless, in other implementations, for example when the system 100 only trains the decoder neural network 102, the system 100 can be supplemented with processing from other systems.

The VAE training framework can utilize unsupervised, semi-supervised, and supervised learning algorithms. When the system 100 does not have access to ground truth knowledge of the conformations and poses of the macromolecule (e.g., exact 3-D atom coordinates), the training engine 106 can use an unsupervised algorithm. For example, in the unsupervised algorithm, the only data obtained by the system 100 is from the set of images 114. In other implementations, the system 100 may receive additional (e.g., ground truth) data and utilize a semi-supervised or supervised algorithm. In some cases, the additional data may originate from highly accurate simulations of a macromolecule that has been tested against experiments.

After training, the system 100 can execute a generating engine 108 to cause the decoder neural network 102 to generate a conformation output 110. The conformation output 110 specifies 3-D atom coordinates 112 of each atom contained in the macromolecule. Using the atom coordinates 112, the system 100 can then determine a macromolecule conformation 304. In addition, the system 100 can impose stereochemical or other constraints directly on the 3-D atom coordinates 112 when generating the conformation of the macromolecule, exploiting prior knowledge about the molecular physics. For example, the system 100 can use prior knowledge of expected bond lengths and a center of mass of the macromolecule to obtain physically plausible conformations. Moreover, using atom coordinates 112 to generate conformations enables shared information between all configurations, across both training and generating procedures of the system 100. This is because conformations defined in coordinate (atom) space can be continuously deformed into one another which is not applicable when processing directly to images.

The generating engine 108 can be executed by the system 100 successively to generate multiple different conformations of the molecule. Hence, after obtaining the set of images 114 depicting the macromolecule in a limited number of conformations and poses, the system 100 generates the, e.g., continuous, distribution of conformations.

Generally, the encoder neural network and the decoder neural network can have any appropriate neural network architectures which enable them to perform their described functions. In particular, the encoder neural network and the decoder neural network can each include any appropriate neural network layers (e.g., fully-connected layer, convolutional layers, attention layers, etc.) in any appropriate numbers (e.g., 5, 10, or 100 layers) and arranged in any appropriate configuration (e.g., as a linear sequence of layers).

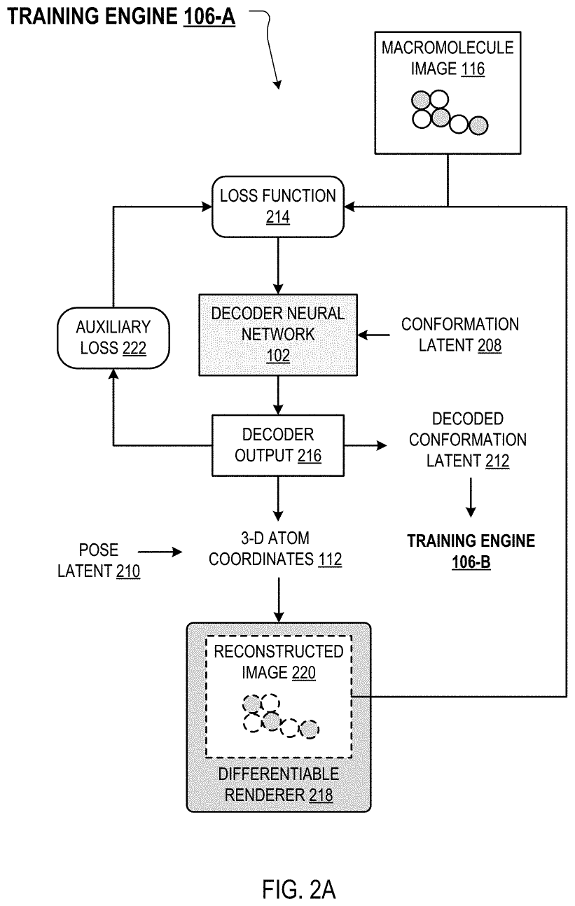

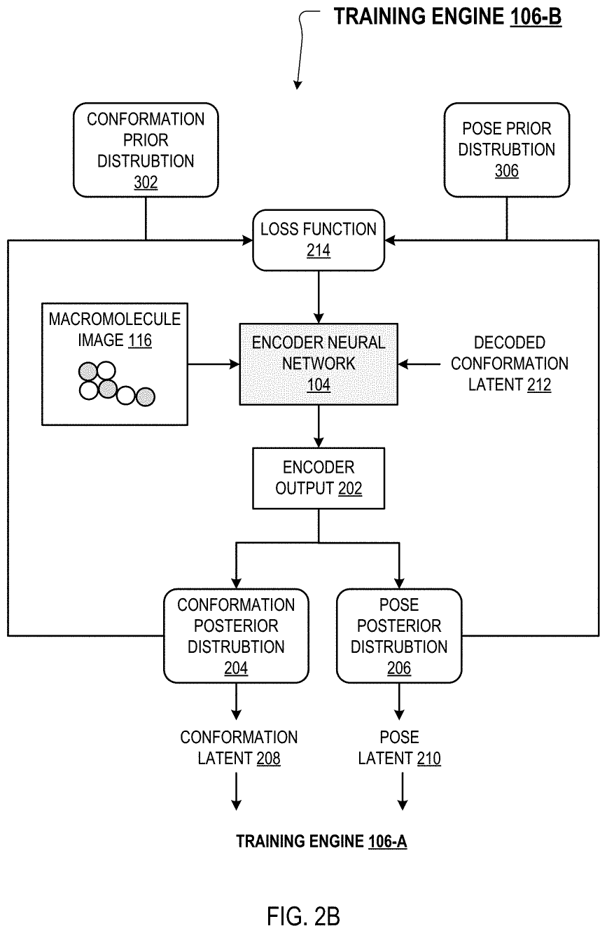

FIG. 2A and FIG. 2B show operations performed by an example training engine 106 using the VAE framework. In this case, a decoder neural network 102 and an encoder neural network 104 are jointly trained on the set of images 114 by the training engine 106.

For demonstrative purposes, the training engine 106 will be decomposed into two subsystems: training engine 106-A (depicted in FIG. 2A) and training engine 106-B (depicted in FIG. 2B) that operate on the decoder 102 and encoder 104, respectively. The decoder 102 and encoder 104 are normally trained in parallel, where processes of the decoder influence the encoder and vice versa. However, in some implementations, the decoder and/or encoder can be trained independently using the training engine 106. In other implementations, only the decoder 102 is trained.

Note, the example training engine 106 is only supplied data from the set of images 114 and thus utilizes an unsupervised learning algorithm, although, as mentioned previously, various learning algorithms are feasible. A general training strategy using VAEs and latent representations will be discussed below, followed by various implementations of the training strategy on the decoder 102 and encoder 104 neural networks.

The VAE implements latent representations (variables in a latent space), e.g., latent random variables, which encode information about conformations and poses of the macromolecule. Conformation latent representations (CLRs) are encoded representations of conformations. That is, they encode the 3-D atom coordinates of unique conformations in a fixed coordinate system. Likewise, pose latent representations (PLRs) are encoded representations of poses. They encode orientations, i.e., translations and/or rotations, of the various conformations with respect to a global coordinate system. The training engine 106 separates the latent representations into two parts corresponding to conformation and pose in order to control these two confounding factors with greater flexibility.

In some implementations, the conformation and pose may be combined into a single latent representation. However, the system 100 benefits from a separated architecture because the neural networks are less susceptible of convergence to degenerate solutions. That is, with the separated architecture, the neural networks are more inclined to reuse conformations in different poses to explain the set of images 114. With a single latent space, the neural networks tend to produce multiple conformations in a single pose only, therefore misinterpreting a conformation in different poses as different conformations.

The latent representations characterize the conformations and poses in lower-dimensional latent spaces, relative to the dimensions of the image data, which can be processed efficiently by the neural networks. Essentially, the latent spaces are compressed data spaces, referred to as information bottlenecks, which extract the most relevant information about conformations and poses from the image set 114. Moreover, the VAE framework captures the distribution of data in the set of images 114 by modelling corresponding distributions over the latent representations. In doing so, the VAE framework can substitute real-world image sampling by sampling latent representations from the latent distributions and then rendering images from the samples. By constraining the neural networks to pass through 3-D atom coordinates, conformations of the molecule can also be generated from the distributions.

Referring to FIG. 2A illustrating operations performed by the training engine 106 to train the decoder neural network 102: The training engine 106 obtains a batch of images from the image set 114. The decoder 102 is then trained on each macromolecule image 116 in the batch using a loss function 214 that characterizes various error metrics to be optimized. In some implementations, the loss function 214 averages the error metrics over all images in the batch, such that the average error of the batch is optimized. In further implementations, the decoder 102 and/or encoder 104 are trained successively on multiple batches of images.

The decoder 102 models a forward process (e.g., decoding) by determining 3-D atom coordinates 112 starting from a CLR 208 and subsequently generating a reconstructed image 220 of the macromolecule from the atom coordinates 112. The reconstructed macromolecule image 220 can be rendered using a differentiable renderer 218. In some cases, the decoder 102 may decode the CLR 208 directly to the reconstructed image 220. However, constraining the decoder 102 to pass through atom coordinates 112 before rendering the reconstruction 218 has several advantages that will be outlined below.

The complete decoding process can involve multiple intermediate steps performed by different layers of the decoder neural network 102. For example, the decoder 102 can receive the CLR 208 at an input layer and translate it into a decoded representation of the CLR 212 at a hidden layer. The decoded CLR 212 can be used by the encoder 104 to autoregressively generate a PLR 210 for the corresponding CLR 208. This process will be described in more detail when referring to FIG. 2B.

In parallel, the decoded CLR 212 can be processed by an output, e.g., linear, layer of the decoder 102 to generate a decoder output 216 that specifies the coordinates 212 for each atom constituting the macromolecule. For example, the decoder output 216 can specify atom coordinates 212 relative to a base conformation of the macromolecule using residue deltas. That is, the decoder output 216 may specify, for each residue in the macromolecule, a translation and rotation of the residue with respect to the base (reference) conformation, and this may be applied to the base conformation to obtain the conformation of the macromolecule. Then, if desired or if needed for subsequent processing, the translated and rotated position of each residue may be used to obtain coordinates for a plurality of atoms of the residue. In this example case, the output layer of the decoder 102 can have 9N dimensions, where N is the total number of residues of the macromolecule. This aspect of the architecture of the system may be selected according to the imaged macromolecule. Each 9-D vector can define a delta describing 3-D translations and rotations of each residue with respect to the base conformation. Three components of the 9-D vector can define a translation vector \vec{t}∈ [symbol] ³, while the remaining six components can define two 3-D rotation vectors \vec{v}₁, \vec{v}₂∈ [symbol] ³. The two rotation vectors can be orthogonalized using a Gram-Schmidt process to obtain a full 3×3 rotation matrix R∈ [symbol] ^{3×3}. Together, the translation vector \vec{t} and rotation matrix R can modify their corresponding residue coordinates to translate and/or rotate the residue with respect to the base conformation. Subsequently, the atoms coordinates 212 can be inferred relative to the modified residue coordinates.

In some implementations, instead of using residues to infer atom positions, the decoder output 216 specifies deltas directly on the atom coordinates 112 relative to atom coordinates of the base conformation. Hence, each atom coordinate is translated and/or rotated with respect to the base conformation. In this case, residues of the macromolecule may not be well-approximated as rigid bodies and therefore each atom may need to be considered individually.

Consequently, in any of these implementations, decoder network parameters θ can parametrize a function ƒ_{θ} that decodes the CLR 208 into atom coordinates x=ƒ_{θ}(z), where z is the CLR 208 and x are the 3-D atom coordinates 112. The function ƒ_{θ} can encompass the various steps performed by different layers of the decoder neural network 102 to generate the atom coordinates 112.

Note that the PLR 210 is generally not decoded by the decoder 102. Here, the PLR 210 is applied directly to the atom coordinates 112 to generate a modified pose of the conformation. For example, the PLR 210 can be an 8-D vector defining a global 2-D translation and 3-D rotation of the conformation in an image plane. Two components of a PLR can define a global 2-D translation vector \vec{T}∈ [symbol] ². The remaining six components of the PLR can define two global 3-D rotation vectors \vec{V}₁, \vec{V}₂∈ [symbol] ³. The two rotation vectors can be orthogonalized using the Gram-Schmidt process to obtain a global rotation matrix [symbol] . Hence, atom coordinates x can be modified by \vec{T} and [symbol] to collectively translate and/or rotate the conformation into the modified pose x′. Consequently, multiple poses of the conformation can be generated by applying multiple PLRs to the atom coordinates 112.

The reconstructed image 220, depicting the conformation in the modified pose, can be rendered as μ_{θ}(z)=render(x′), where ‘render’ denotes computations of the differentiable renderer 218 and μ_{θ}(z) represents the reconstructed image 220. The advantage of constraining the decoder 102 to pass through atom coordinates 112 before rendering the reconstructed image 220 is twofold: (i) prior knowledge of the molecular physics can be imparted to the reconstruction 220 which limits the space of possible reconstructions and (ii) after training, atom coordinates 112 corresponding to conformations can be directly generated from the decoder 102 simply by sampling CLRs.

In some implementations, the differentiable renderer 218 models the image capture process used to obtain the image 116 when rendering the reconstructed image 220. In doing so, the reconstructed image 220 can include noise and various effects that alter image quality. For example, if the image 116 is from a micrograph, the renderer 218 can model the cryo-EM process. The renderer 218 can accomplish this by using a projection of an electron density of the macromolecule followed by a convolution of a Contrast Transfer Function (CTF). The CTF mathematically describes how aberrations in the EM process modify the macromolecule image 116. In this case, the electron density for each heavy atom in the macromolecule can be modeled by a single Gaussian blob with a standard deviation of 1 Å and unit mass. Accordingly, rendering functions can be computed analytically by directly projecting to each output image pixel. Moreover, this model has projection operators that are end-to-end differentiable which can be beneficial for training, e.g., when passing gradients through the model.

Note that the decoder 102 generally receives a set of CLRs for each image 116, producing a respective decoder output 216 for each CLR 208 in the set. The resulting atom coordinates 112 of each decoder output 216 can then be modified by one or more respective PLRs. Hence, multiple reconstructed images, depicting multiple conformations in various poses, may be rendered by the differentiable renderer 218. The training engine 106 can compare reconstructed images with the given image 116 to determine how well a particular conformation in a particular pose explains the image data.

Specifically, the training engine 106 can train the neural networks on these reconstructed images using the loss function 214. For example, the loss function 214 can include terms that measure a reconstruction error between the macromolecule image 116 and all reconstructed images. Accordingly, the training engine 106 can optimize the loss function 214 with respect to the decoder parameters θ (e.g., using a stochastic gradient descent method) such that the conformations decoded from the CLRs x=ƒ_{θ}(z), with poses modified by the PLRs, provide the best explanation of the image data.

In some implementations, the reconstruction error measures an average (sum) of image likelihoods, where an image likelihood P_{θ}(y|z) is the probability of the image 116 (y) given the CLR 208 (z). Hence, optimizing the loss function 214 can amount to maximizing the average of image likelihoods over all reconstructions of the image 116. For example, the training engine 106 can model the image likelihood as a normal (Gaussian) distribution P_{θ}(y|z)= [symbol] (y:μ_{θ}(z),σ_{θ}(z)), where a mean image μ_{θ}(z) and an image variance σ_{θ}(z) parameterize the distribution. In this case, the mean image μ_{θ}(z) can be identified with the reconstructed image 220 generated from the differentiable renderer 218. The image variance σ_{θ}(z) can be dependent or independent of the CLR 208. In some implementations, the image variance is set to the noise of the image capture process that the differentiable renderer 218 is simulating, for example, the noise in the cyro-EM process.

In other implementations, the reconstruction error measures an average of image log-likelihoods, i.e., a product of image likelihoods. However, this type of reconstruction error can discourage the neural networks from exploring conformations as low-probability CLRs are penalized strongly.

In further implementations, the loss function 214 includes auxiliary loss 222 that measure, for each decoder output 216 specifying the atom coordinates 112 of a conformation, a deviation of a structure of the macromolecule from an expected structure of the macromolecule. For example, the structure specified by the 3-D atom coordinates 112 can be compared with expected values. For example, the auxiliary loss 222 can include a term that measures a deviation (e.g., mean squared deviation) between bond lengths along a backbone of the macromolecule and expected bond lengths along the backbone. This term can train the neural networks towards physically plausible predictions. Alternatively or in addition, the auxiliary loss 222 can include a term that measures a deviation between a center of mass of the macromolecule and an expected center of mass. This term can keep the macromolecule structure centered on zero, forcing the neural networks to represent translations using PLRs instead of translating all atoms and/or residues independently.

Note that, in principle, the decoder 102 can be solely trained on the error metrics of the aforementioned loss function 214, without introducing the encoder 104. For instance, each CLR 208 can be sampled from a prior distribution over CLRs z˜P_{θ}(z) while the PLR 210 can be sampled from a prior distribution over PLRs z′˜P_{θ}(z′), where primed variables denote PLRs. Generally, the priors are modeled by the training engine 106 as standard normal distributions, which in terms of the pose, corresponds to a normal distribution of 2-D translations about a center and a uniform distribution of 3-D rotations. However, training the decoder 102 using this approach can require a prohibitively large number of samples and is therefore computationally expensive. The training engine 106 introduces the encoder 104 to speed up the process.

Referring now to FIG. 2B that illustrates operations performed by the training engine 106 to train the encoder neural network 104. The encoder 104 is trained on the same image 116 and loss function 214 as the decoder 102, i.e., the encoder 104 and decoder 102 may be trained jointly. Hence, the loss function 214 can be optimized with respect to both decoder θ and encoder ϕ network parameters.

The encoder 104 models an inverse process (e.g., encoding) by generating posterior distributions over CLRs 204 and PLRs 206 starting from the macromolecule image 116. Specifically, the encoder 104 processes the image 116 to generate an encoder output 202 that specifies parameters defining the posterior distributions over the latent representations 204/206. The encoder 104 can also process the decoded CLR 212 when generating the encoder output 202, which can therefore be used to autoregressively specify the parameters of the PLR posterior 206.

For example, the encoder 104 can be split into three neural networks: (i) an image encoder that encodes the image 116 into an encoded representation of the image, (ii) a conformation encoder that processes the encoded representation of the image to generate the parameters of the CLR posterior 204 and (iii) a pose encoder that processes the encoded representation of the image and, in implementations, the decoded CLR 212 to autoregressively generate the parameters of the PLR posterior 206.

Subsequently, the training engine 106 can supply CLRs 208 and PLRs 210 to the decoder 102 by sampling from their respective posterior distributions 204/206. Following the steps mentioned previously, the decoder 102 can process the CLRs 208 to generate respective atom coordinates 112 and modify them with their corresponding PLRs 210. Reconstructed images 220 of the conformations in modified poses can then be generated by the differentiable renderer 218.

In some implementations, the encoder 104 models the posteriors as normal distributions such that the parameters of the posterior distributions are means and variances. For example, the CLR posterior 204 can be modelled by the encoder 104 as Q_{ϕ}(z|y)= [symbol] (z;μ_{ϕ}(y),σ_{ϕ}(y)), where μ_{ϕ}(y) is a mean CLR and σ_{ϕ}(y) is a CLR variance. Similarly, the PLR posterior 206, conditioned on the decoded CLR 212, can be modelled by the encoder 104 as Q_{ϕ}(z′|y, z)= [symbol] (z′;μ_{ϕ}(y,z),σ_{ϕ}(y,z)), where μ_{ϕ}(y,z) is a mean PLR and σ_{ϕ}(y,z) is a PLR variance.

Note that the parameters of the posterior distributions 204/206 are themselves parametrized by the encoder network parameters ϕ. Therefore, the encoder 104 can be trained on the loss function 214 to match the posterior distributions 204/206 with the prior distributions, P_{θ}(z) and P_{θ}(z′), such that samples drawn from the posteriors 204/206 are, approximately, statistically independent of the set of images 116. In parallel, the decoder 102 efficiently learns to decode unconditional samples of CLRs.

For example, the loss function 214 can include terms that measure a divergence (e.g., Kullback-Leibler divergence) between the posterior distributions (as defined by the parameters generated by the encoder output) and the prior distributions, to maximize the similarity between the distributions. Taking into account the other error metrics, the loss function 214 can control tradeoffs between image reconstruction error and posterior/prior distribution matching, as well as auxiliary loss 222, by weighting different error terms. As mentioned previously, the loss function 214 generally averages the error over all images in the batch of images. Hence, the training engine 106 conducts the abovementioned process for each image in the batch.

In some implementations, the training engine 106 can conduct an initial phase of pose-only training by strictly predicting poses of a base conformation of the macromolecule. That is, the training engine 106 first trains the neural networks using PLR samples to modify poses of the base conformation. After the neural networks converge, the training engine 106 can begin predicting conformations by training the neural networks on CLR samples.

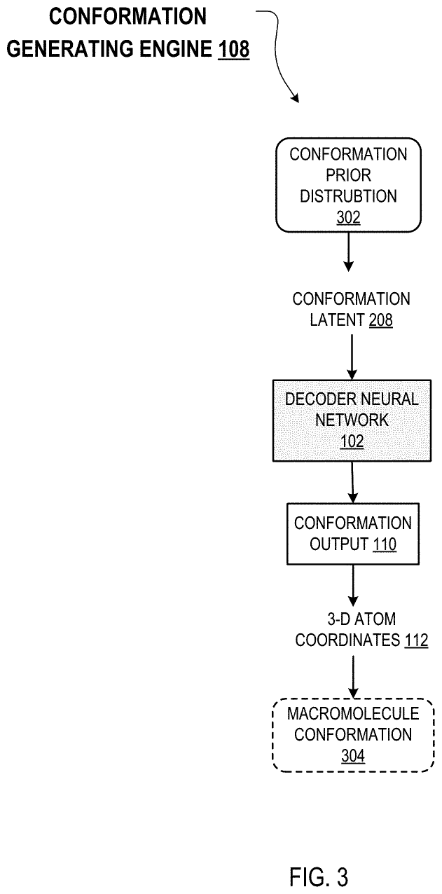

Referring to FIG. 3 that illustrates operations performed by an example generating engine 108 after the decoder neural network 102 is trained by the training engine 106.

The generating engine 106 can sample a CLR 208 from the CLR prior 302. The CLR 208 is processed by the decoder 102 to generate a conformation output 110 that specifies 3-D atom coordinates 112 for each atom constituting the macromolecule. Subsequently, the generating engine 108 can determine a macromolecule conformation 304 from the conformation output 110 using the atom coordinates 112.

In some implementations, the conformation output 110 specifies a delta for the atom coordinates 112 relative to coordinates in a base conformation of the molecule. The generating engine 108 can apply the delta to the base conformation coordinates to determine the conformation 304. In some implementations the base conformation is determined from a single state (conformation) reconstruction, e.g., from a (single) conformation of the molecule determined by conventional means such as x-ray crystallography, or determined from computational modelling, or determined in some other way.

In other implementations, the conformation output 110 specifies a delta for residues relative to positions of the residue in the base conformation of the molecule. In this case, the delta can define translations and/or rotations of the residue with respect to the base conformation positions. The generating engine 108 can infer the atom coordinates 112 from the residues positions.

The generating engine 108 can be executed successively to generate multiple conformations of the macromolecule.

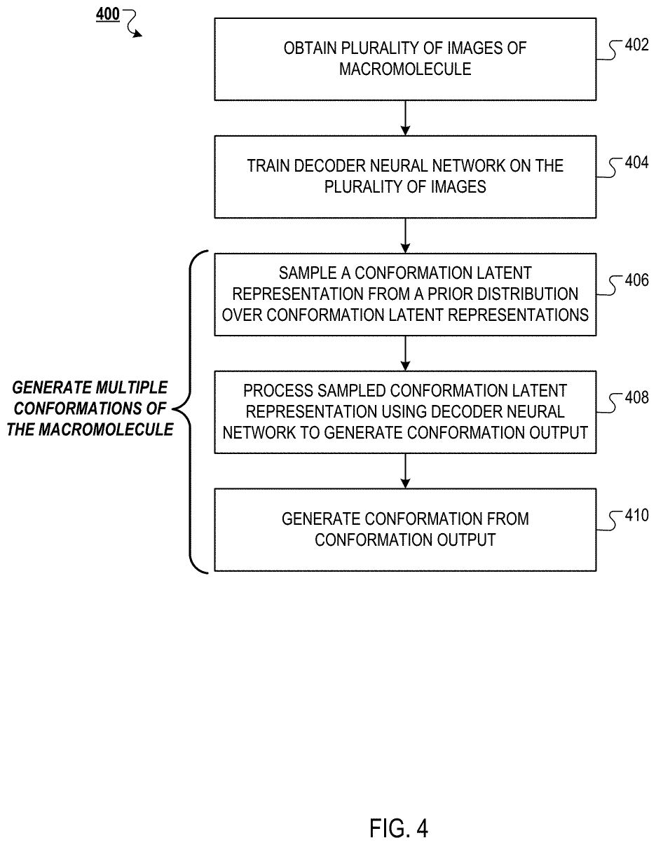

FIG. 4 is a flow diagram of an example process 400 for generating multiple conformations of a macromolecule from a decoder neural network. For convenience, the process 400 will be described as being performed by a system of one or more computers located in one or more locations. For example, a conformation prediction system, e.g., the conformation prediction system 100 of FIG. 1, appropriately programmed in accordance with this specification, can perform the process 400.

The system obtains a plurality of images of a macromolecule (402). The macromolecule can be a large molecule composed of a plurality of atoms. For example, the macromolecule can be a protein, an amino-acid, a nucleic-acid, a carbohydrate, a lipid, a nanogel, a macrocycle, etc. The plurality of images can be obtained from any suitable imaging system (e.g., cryo-EM).

The system trains a decoder neural network on the plurality of images of the macromolecule (404).

The system samples a conformation latent representation from a prior distribution over conformation latent representations (406). The prior distribution may be any distribution, e.g., a standard normal distribution. During training of the decoder neural network a (posterior) distribution of the conformation latent representation processed by the decoder neural network and the prior distribution may be encouraged to be similar, e.g., by an objective function used during the training.

The system processes the conformation latent representation using the decoder neural network to generate a conformation output (408).

The system generates a macromolecule conformation from the conformation output (410).

The system can repeat steps 406-410 to generate multiple conformations of the macromolecule.

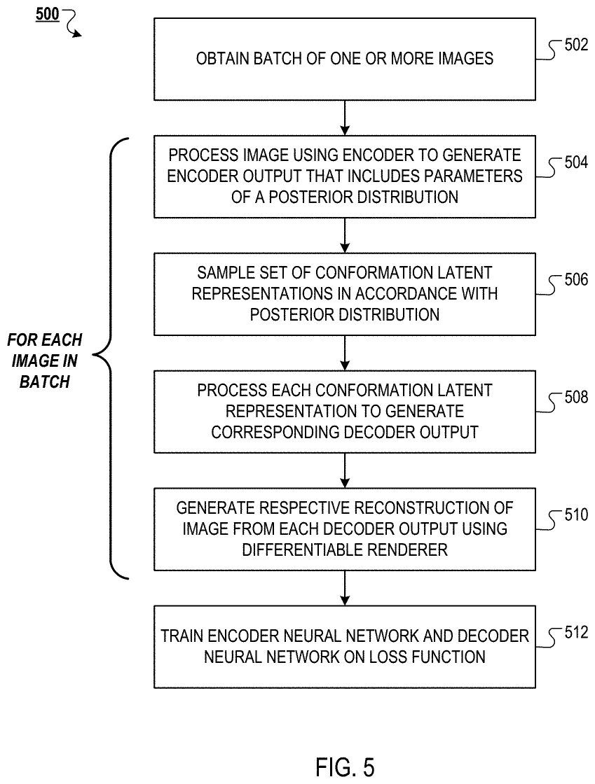

FIG. 5 is a flow diagram of an example process 500 for jointly training a decoder neural network and an encoder neural network. For convenience, the process 500 will be described as being performed by a system of one or more computers located in one or more locations. For example, a conformation prediction system, e.g., the conformation prediction system 100 of FIG. 1, appropriately programmed in accordance with this specification, can perform the process 500.

The system obtains a batch of one or more images (502). The batch of images can be obtained from the plurality of images.

The system processes an image from the batch using an encoder neural network to generate an encoder output that includes parameters of a posterior distribution over conformation latent representations (504). As described below, the posterior distribution may be sampled from to obtain a conformation latent representation that is sampled from to generate the conformation output from the decoder.

The system samples a set of conformation latent representations from the posterior distribution over conformation latent representations (506).

The system processes each conformation latent representation in the set to generate a corresponding decoder output (508).

The system generates a respective reconstruction of the image from each decoder output using a differential renderer (510).

The system repeats steps 504-510 for each image in the batch to generate a set of reconstructed images for each respective image.

The system trains the encoder neural network and decoder neural network on a loss function that includes terms that measure an error between the images and their respect set of reconstructed images (512).

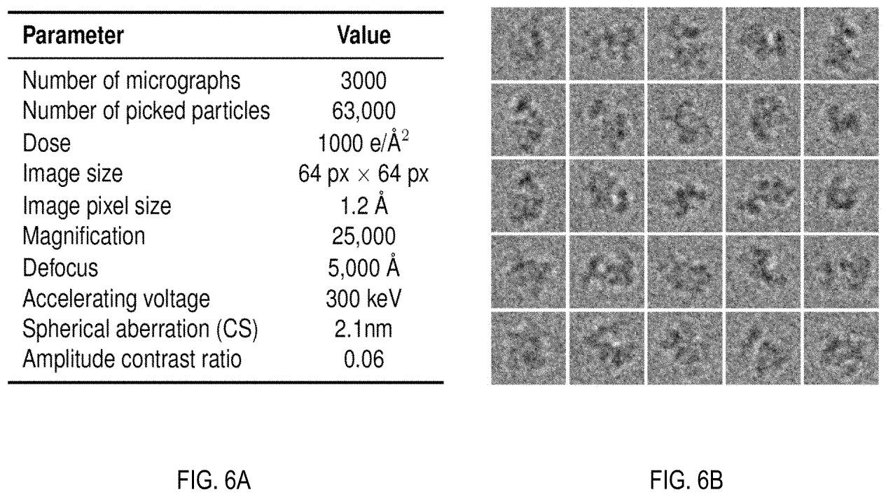

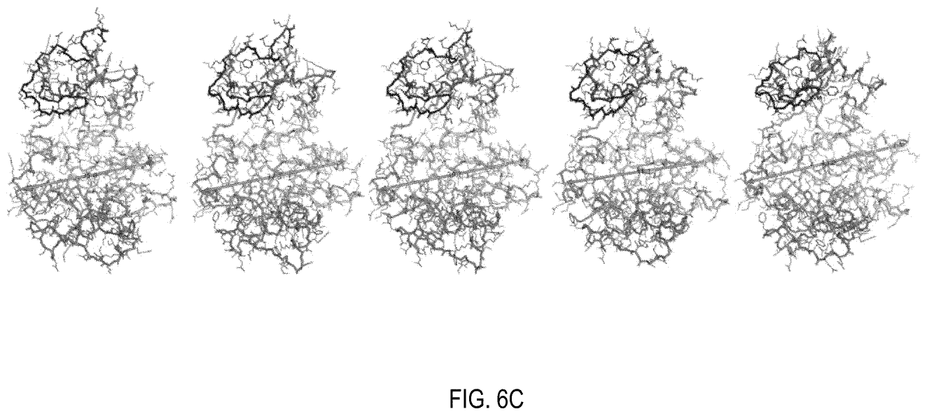

FIG. 6A is a table showing parameters of a cryo-EM simulator used for experimentation. The cryo-EM simulator can be used to test the performance of the conformation prediction system 100 of FIG. 1 by comparing predicted conformations and distributions with ground truth data produced by the simulator. Details of a particular experiment are outlined below.

As mentioned previously, realistic ground truth data can be produced using an externally validated Molecular Dynamics (MD) simulation, as well as simulating the image formation process using a high quality simulator including, e.g., a contrast transfer function, realistic noise models and solvation. In this case, conformations were selected from a trajectory of Aurora A Kinase (AurA) in an apo (unbound) state, generated with MD simulations by Folding@Home. Details of which are described by S. Cyphers, E. F. Ruff, J. M. Behr, J. D. Chodera and, N. M. Levinson in “A water-mediated allosteric network governs activation of aurora kinase a.” in Nature Chemical Biology, 13(4):402, 2017.

AurA is a non-membrane monomeric enzyme with flexible catalytic and activation loops. 3000 sampled conformations served as input to simulate the cryo-EM imaging process, using a TEM-simulator to realistically model the image formation and noise of a real cryo electron microscope. Details of the TEM-simulator are described by H. Rullgård, L.-G. Öfverstedt, S. Masich, B. Daneholt, and O. Öktem in “Simulation of transmission electron microscope images of biological specimens” in Journal of Microscopy, 243(3):234-256, 2011.

Each sampled conformation was placed randomly (rotation and translation) on a resulting micrograph. AurA has a size (33 kDa, 282 residues) below current practical experimental limits of cryo-EM but was selected due to limited options of highly-dynamic MD on large proteins. Therefore, a ˜10 times higher-than-usual electron dose (√10˜3 times higher SNR) was applied to make the difficulty of determining poses comparable to a protein of about 1000 residues. Otherwise, the simulation setup broadly follows standard practice.

FIG. 6B shows a random sample of picked particle images used as input to the conformation prediction system 100. FIG. 6C shows examples of conformations of AurA, illustrating changes in molecular conformation as the conformation latent representation input to the decoder neural network changes, and showing that a range of different conformations can be obtained by sampling from the prior distribution. The double-headed arrow indicates the T288Cα-R255Cζ distance, which changes as the protein changes from a “short state” (left) to a “long state” (right).

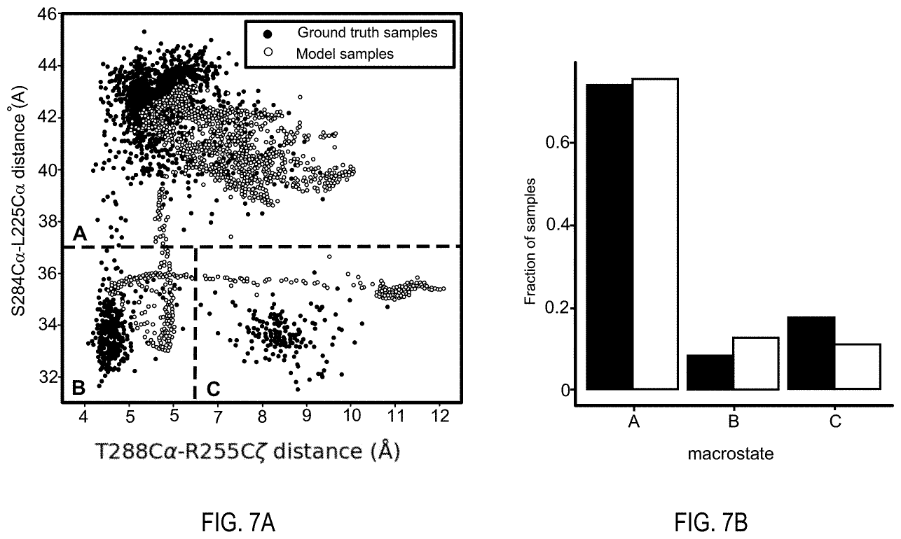

FIG. 7A shows a distribution of the ground truth MD samples compared to predicted distributions reconstructed by the system 100 over two distances pairs of AurA, S284Cα-L225Cα and T288Cα-R255Cζ. Sample quality is illustrated by representing each protein structure as a point defined by the two pairwise distances. Dashed lines stratify samples into groups (A, B, and C) using 6.5 Å as a cutoff on the X-axis and 37 Å as a cutoff on the Y-axis.

FIG. 7B shows the portion of samples in each of the groups (A, B, and C). All three major modes seen in this projection are successfully recovered by conformation prediction system 100, including correctly determining their approximate relative distributions.

Merely as one example, FIGS. 6 and 7 were generated using a encoder neural network 104 comprising an MLP, in particular a four layer MLP (multilayer perceptron) that processed a flattened version of image 116, to generate the encoded representation of the image. The parameters of the CLR posterior 204 were generated from the encoded representation of the image using a conformation encoder comprising a layer norm and a linear layer. The parameters of the PLR posterior 206 were generated from the encoded representation of the image using a layer norm and a linear layer. The decoded CLR 212 was added to this (summed) and the combination processed by an MLP, in particular a three layer MLP. The decoder neural network 102 comprised a residual network (a ResNet with 5 blocks) followed by a layer norm and a linear layer; a further linear layer generated a 9-D vector as described above.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a standalone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a sub combination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
obtaining a plurality of images of a macromolecule having a plurality of atoms;
training a decoder neural network on the plurality of images, wherein the decoder neural network is configured to receive an input comprising a conformation latent representation of a conformation of the macromolecule and to process the input to generate a conformation output that specifies three-dimensional coordinates of each of the plurality of atoms; and
after the training, generating a plurality of conformations for at least a portion of the macromolecule that each include respective three-dimensional coordinates of each of the plurality of atoms, comprising, for each conformation:
sampling a conformation latent representation from a prior distribution over conformation latent representations;
processing a respective input comprising the sampled conformation latent representation using the decoder neural network to generate a conformation output that specifies three-dimensional coordinates of each of the plurality of atoms for the conformation; and
generating the conformation from the conformation output.

2. The method of claim 1, wherein the conformation output specifies, for each of the plurality of atoms, a respective delta for base three-dimensional coordinates for the atom in a base conformation for the macromolecule.

3. The method of claim 2, wherein generating the conformation from the conformation output comprises:
for each of the plurality of atoms, applying the respective delta specified by the conformation output for the atom to the base three-dimensional coordinates for the atom to generate the respective three-dimensional coordinates for the atom.

4. The method of claim 2, further comprising:
determining the base conformation for the macromolecule through a single state reconstruction.

5. The method of claim 2, wherein the delta specifies, for each of a plurality of residues that each include one or more of the plurality of atoms, a respective relative translation and relative rotation for the residue relative to a position of the residue in the base conformation.

6. The method of claim 1, wherein training the decoder neural network on the plurality of images comprises:
training the decoder neural network jointly with an encoder neural network that is configured to receive an image of the macromolecule and to process the image to generate an encoder output that comprises parameters of a posterior distribution over the conformation latent representations.

7. The method of claim 6, wherein training the decoder neural network jointly with the encoder neural network comprises:
obtaining a batch of one or more images from the plurality of images;
for each image in the batch:
processing the image using the encoder neural network to generate an encoder output;
sampling a set of conformation latent representations from the posterior distribution in accordance with the parameters of the posterior distribution in the encoder output;
processing each of the conformation latent representations in the set using the decoder neural network to generate a respective decoder output for each of the conformation latent representations;
generating a respective reconstruction of the image from each of the decoder outputs using a differentiable renderer; and
training the encoder neural network and the decoder neural network on a loss function that includes one or more loss terms that measure, for each image in the batch, an error between the image and the respective reconstructions of the image generated from the decoder output for the image.

8. The method of claim 7, wherein the set of conformation latent representations includes a plurality of conformation latent representations.

9. The method of claim 7, wherein the loss function includes one or more auxiliary loss terms that measure, for each decoder output, a deviation of a structure of the macromolecule as specified by the three-dimensional coordinates of each of the plurality of atoms from an expected structure of the macromolecule.

10. The method of claim 9, wherein the auxiliary loss terms include a first auxiliary loss term that measures a deviation between (i) bond lengths along a backbone of the macromolecule in the structure specified by the three-dimensional coordinates of each of the plurality of atoms and (ii) expected bond lengths along the backbone of the macromolecule.

11. The method of claim 9, wherein the auxiliary loss terms include a second auxiliary loss term that measures a deviation between (i) a center of mass of the structure specified by the three-dimensional coordinates of each of the plurality of atoms and (ii) an expected center of mass of the structure.

12. The method of claim 7, wherein the loss function includes one or more terms that measure, for each encoder output, a divergence between the posterior distribution and the prior distribution in accordance with the parameters specified in the encoder output.

13. The method of claim 7, wherein:
the encoder neural network is configured to process the image to generate an encoded representation of the image and to process the encoded representation of the image to generate the parameters of the posterior distribution over the conformation latent representations;
the encoder neural network is configured to process at least the encoded representation to generate parameters of a posterior distribution over pose latent representations; and
generating a respective reconstruction of the image from each of the decoder outputs using a differentiable renderer comprises:
sampling a pose latent representation from the posterior distribution over pose latent representations in accordance with the parameters of the posterior distribution; and
for each decoder output, generating the respective reconstruction of the image using the sampled pose latent representation and the differentiable renderer.

14. The method of claim 13, wherein generating the respective reconstruction of the image using the sampled pose latent representation and the differentiable renderer comprises:
generating, from the decoder output, three-dimensional coordinates of each of the plurality of atoms;
modifying a pose of the plurality of atoms using the sampled pose latent to generate modified three-dimensional coordinates of each of the plurality of atoms; and
applying the differentiable renderer to the modified three-dimensional coordinates of each of the plurality of atoms to generate the respective reconstruction.

15. The method of claim 13, wherein the decoder neural network is configured to process the input to generate a decoded representation of the conformation latent representation and to process the decoded representation to generate the decoder output, and wherein the encoder neural network is configured to process the encoded representation of the image and respective decoded representation of each conformation latent representation in the set to generate the parameters of the posterior distribution over pose latent representations.

16. The method of claim 1, wherein the plurality of images are Cryo-electron microscopy (cryo-EM) images of the macromolecule.

17. The method of claim 1, wherein the plurality of images are picked particle images of the macromolecule.

18. The method of claim 1, wherein the macromolecule is a protein.

19. A system comprising one or more computers and one or more storage devices storing instructions that when executed by the one or more computers cause the one more computers to perform operations comprising:
obtaining a plurality of images of a macromolecule having a plurality of atoms;
training a decoder neural network on the plurality of images, wherein the decoder neural network is configured to receive an input comprising a conformation latent representation of a conformation of the macromolecule and to process the input to generate a conformation output that specifies three-dimensional coordinates of each of the plurality of atoms; and
after the training, generating a plurality of conformations for at least a portion of the macromolecule that each include respective three-dimensional coordinates of each of the plurality of atoms, comprising, for each conformation:
sampling a conformation latent representation from a prior distribution over conformation latent representations;
processing a respective input comprising the sampled conformation latent representation using the decoder neural network to generate a conformation output that specifies three-dimensional coordinates of each of the plurality of atoms for the conformation; and
generating the conformation from the conformation output.

20. One or more computer storage media storing instructions that when executed by one or more computers cause the one more computers to perform operations comprising:
obtaining a plurality of images of a macromolecule having a plurality of atoms;
training a decoder neural network on the plurality of images, wherein the decoder neural network is configured to receive an input comprising a conformation latent representation of a conformation of the macromolecule and to process the input to generate a conformation output that specifies three-dimensional coordinates of each of the plurality of atoms; and
after the training, generating a plurality of conformations for at least a portion of the macromolecule that each include respective three-dimensional coordinates of each of the plurality of atoms, comprising, for each conformation:
sampling a conformation latent representation from a prior distribution over conformation latent representations;
processing a respective input comprising the sampled conformation latent representation using the decoder neural network to generate a conformation output that specifies three-dimensional coordinates of each of the plurality of atoms for the conformation; and
generating the conformation from the conformation output.

21. A non-transitory computer storage medium storing data that defines a plurality of conformations for at least a portion of a macromolecule, wherein the data was generated by operations comprising:
obtaining a plurality of images of a macromolecule having a plurality of atoms;
training a decoder neural network on the plurality of images, wherein the decoder neural network is configured to receive an input comprising a conformation latent representation of a conformation of the macromolecule and to process the input to generate a conformation output that specifies three-dimensional coordinates of each of the plurality of atoms; and
after the training, generating a plurality of conformations for at least a portion of the macromolecule that each include respective three-dimensional coordinates of each of the plurality of atoms, comprising, for each conformation:
sampling a conformation latent representation from a prior distribution over conformation latent representations;
processing a respective input comprising the sampled conformation latent representation using the decoder neural network to generate a conformation output that specifies three-dimensional coordinates of each of the plurality of atoms for the conformation; and
generating the conformation from the conformation output.
